(19) United States
(12) Patent Application Publication
(10) Pub. No.: US 2025/0320031 A1
(43) Pub. Date: Oct. 16, 2025

(54) CARTRIDGE NEST, WHICH FEATURES INTEGRATED CENTERING OF THE CARTRIDGES

(71) Applicant: SCHOTT Pharma Schweiz AG, St. Gallen (CH)

(72) Inventors: Nithan Vasanthamohan, St. Gallen (CH); Marcel Berlinger, St. Gallen (CH)

(21) Appl. No.: 19/172,380

(22) Filed: Apr. 7, 2025

(30) Foreign Application Priority Data
Apr. 15, 2024 (EP) 24170195.2

(51) Int. Cl. B65D 25/10; A61J 1/16; B65B 3/00; B65D 77/04

(52) U.S. Cl. CPC B65D 25/108; A61J 1/16; B65B 3/003; B65D 77/0446; B65D 2577/043

(57) ABSTRACT

A holding structure for concurrently holding a plurality of primary packaging containers for substances for pharmaceutical, medical or cosmetic applications, the holding structure including a plurality of receptacles for accommodating at least a section of the elongated cylindrical body region of the primary packaging container, each receptacle having a depth, an upper end for inserting the primary packaging container into the receptacle, a bottom end having a holding portion configured to limit an axial movement of the primary packaging container in the receptacle, a circumferentially formed side wall extending in a longitudinal direction from the upper end to the bottom end and a longitudinal axis, alignment elements in each receptacle projecting radially into the receptacle to contact the outer surface of the primary packaging container; wherein the alignment elements arranged so that a primary packaging container accommodated in the receptacle in a resting position is tilted by an angle α of not more than 2.4 degrees.

This claims the priority to European Patent application EP24170195.2, filed on Apr. 15, 2024 and hereby incorporated by reference herein.

The present invention relates to a holding structure for concurrently holding a plurality of containers.

### BACKGROUND

Containers made from glass and later on also from polymer have been applied for storing fluids and powders safely for a long time. In the last decades, the arts in which glass and polymer containers are used for transporting fluids and powders have become increasingly diverse and sophisticated. One such art is the technical field of the present application: pharmaceutical packaging. In the pharmaceutical industry, containers—such as vials, syringes, ampoules, and cartridges—are used as primary packaging for all kinds of pharmaceutically relevant compositions, in particular drugs, such as parenteralia including vaccines, and also for cosmetical compositions, in particular cosmetical compositions which are to be injected into the skin.

In the processing of containers for use in pharmaceutical or cosmetical applications, generally so-called nested solutions are often preferred nowadays, where a holding structure for containers (also referred to as nest) is used for concurrently holding or supporting a plurality of primary packaging containers in a given configuration. An example of a known nested solution is commercially available from Schott Pharma AG under the tradename SCHOTT iQ® platform. The nest with the primary packaging containers is usually delivered to a customer, such as a pharmaceutical company or filler, packaged in a transport or packaging container (also referred to as tub). For further processing the primary packaging containers, the tub is opened. Further processing the primary packaging containers often includes automated steps of removing the primary packaging containers from the tub; filling the primary packaging containers with a composition, e.g. a pharmaceutical or cosmetical composition; and closing the pre-filled primary packaging containers.

### SUMMARY OF THE INVENTION

For filling the containers accommodated in the holding structures filling nozzles are used that are, to a certain extent, inserted into the opening of the containers. In such an automated filling process the containers must be positioned as parallel to the axis of the filling nozzles as possible. If this is not the case and the container is positioned at a slight angle, fill level fluctuations or even damage to the filling nozzle can occur if the filling needle collides with the inclined container. Also, an incorrect positioning of the containers can also lead to failures or errors when lifting the container before they are filled as the crimp can get stuck on the holding feature.

In automated filling processes of the prior art a separate positioning aid in the form of a perforated plate is used that guarantees a parallel positioning of each container accommodated in the supporting structure. Such a position aid, however, not only represents an additional component that is needed in the automated filling process but may also be problematic as the introduction of the open ends of the containers into the perforated plate is made more difficult if the containers are tilt to a certain extend in the holding structure. Also, handling of machine components such as perforated plates above the open containers which may already comprise the medicament can also lead to contamination within the filled container. Finally, depending on the material quality of the perforated plate that is commonly used to ensure a proper alignment of the containers, friction between the perforated plate and the container may cause abrasion on the glass surface and thus damage of the container.

In general, it is an object of the present invention to at least partly overcome a disadvantage arising from the prior art.

It is an object of the present invention to provide a nested solution of pharmaceutical, medical, or cosmetic containers which allows filling of the containers accommodated in the nest as efficiently as possible without the necessity of using separate positioning means. More particularly, the nested solution should allow filling of the containers accommodated in the nest with no damage of the containers by the needle used to fill the container and with as little fill level fluctuations as possible, even if no additional separate positioning means are used. Also, the nested solution should allow the packaging containers to be inserted into and removed from the nest with as little abrasion of the outer surface of the packaging containers as possible.

|1a| A contribution to solving at least one of the objects according to the invention is made by a 1^{st} embodiment of a holding structure for concurrently holding a plurality of primary packaging containers for substances for pharmaceutical, medical or cosmetic applications, each primary packaging container comprising an elongated cylindrical body region having a longitudinal axis L_{container} that runs in a longitudinal direction through the center of the elongated cylindrical body region, the holding structure comprising:
a plurality of receptacles for accommodating at least a section of the elongated cylindrical body region of the primary packaging container, each receptacle having a depth h, an upper end for inserting the primary packaging container into the receptacle, a bottom end having a holding portion configured to limit an axial movement of the primary packaging container in the receptacle, a circumferentially formed side wall extending in a longitudinal direction from the upper end to the bottom end and a longitudinal axis L_{receptable} that runs in a longitudinal direction through the center of the receptacle;
alignment elements in each receptacle that project radially into the receptacle and that are adapted and arranged to contact a part of the outer surface of the elongated cylindrical body region of a primary packaging container accommodated in the receptacle;
   wherein the alignment elements in each receptacle are adapted and arranged to contact a part of the outer surface of the elongated cylindrical body region in such a way that a primary packaging container accommodated in the receptacle in a resting position is tilted by an angle α of not more than 2.4 degrees, preferably not more than 2.2 degrees, more preferably not more than 2.0 degrees, even more preferably not more than 1.8 degrees, even more preferably not more than 1.6 degrees, even more preferably not more than 1.4 degrees, even more preferably not more than 1.2 degrees, and most preferably not more than 1 degree, the tilting angle α being the angle that is formed between longitudinal axis L_{receptacle} and longitudinal axis L_{container} of the primary packaging container accommodated in the receptacle and the resting position being the position in which the holding portion limits the axial movement of the primary packaging container accommodated in the in the receptacle. Most preferably, a primary packaging container accommodated in the receptacle in a resting position is not tilted at all, i.e., α is 0 degrees.

The term “a plurality of receptacles for receiving the primary packaging containers therein at least in sections” as used herein indicates that the depth h of the receptacle is smaller than the height of the primary packaging container to be accommodated in the receptacle so that in the resting position portions of the primary packing container protrude from the receptacle.

To ensure that the primary packaging containers in the holding structure are tilted as little as possible in the receptacles the skilled person would have considered increasing the guide length and therefore the depth of the receptacles or reducing the internal diameter of the receptacles in which the primary packaging containers are accommodated. However, these measures would lead to particle generation in the manufacturing process, i.e. in the injection molding process, due to abrasion during the demolding process. In addition, inserting and removing the primary packaging containers into and from the holding structure with a large contact surface and low pocket internal diameter can lead to various defects, such as the generation of particles, the destruction of the primary packaging container surface or a jamming of the primary packaging containers in the holding structure.

Surprisingly, however, it has been observed that an as vertical possible alignment of the primary packaging containers in the holding structure can be ensured by the provision of alignment elements that are arranged in the receptacles and that contact only a part of the outer surface of the primary packaging containers, more precisely only a part of the outer surface of the elongated cylindrical body region of these containers. Thus, to achieve the parallel alignment of the primary packaging containers in the holding structure it is neither necessary to increase the depth of the receptacles nor to decrease the diameter of the receptacles or to use separate positioning means (although such positioning means may nevertheless be used as the alignment elements also facilitate the introduction of the containers in the holes of the positioning means).

|2a| According to a preferred embodiment of the holding structure according to the present invention, the alignment elements in each receptacle are adapted and arranged to contact a part of the outer surface of the elongated cylindrical body region in such a way that a primary packaging container accommodated in the receptacle in a lifting position is still tilted by an angle α′ of not more than 2.4 degrees, preferably not more than 2.2 degrees, more preferably not more than 2.0 degrees, even more preferably not more than 1.8 degrees, even more preferably not more than 1.6 degrees, even more preferably not more than 1.4 degrees, even more preferably not more than 1.2 degrees and most preferably not more than 1.0 degree, the tilting angle α′ being the angle that is formed between longitudinal axis L_{receptacle} and longitudinal axis L_{container} of the primary packaging container accommodated in the receptacle and the lifting position being the position in which the primary packaging container is lifted out of the resting position in a direction away from the bottom end to a lifting height a, wherein a is 10 to 50%, preferably 20 to 48% and more preferably 25 to 45% of the depth h of the receptacle. Most preferably, also in the lifting position a primary packaging container accommodated in the receptacle is not tilt at all, i.e., α′ is 0 degrees. Preferably, a is in the range from 13 to 20 mm. This embodiment is a 2^{nd} embodiment of the holding structure according to the present invention, that preferably depends on the 1^{st} embodiment.

|3a| According to a further preferred embodiment of the holding structure according to the present invention, holding protrusions acting as holding portions are provided at the bottom ends of the receptacles, which extend radially inwards into the receptacles. Each receptacle may, for example, have two holding protrusions which are diametrically opposite to each other (as shown in FIGS. 3 and 4). In principle, a single holding protrusion is also sufficient for limiting an axial movement of the primary packaging containers in the receptacles, which may also be designed to be circumferential or essentially circumferential. This embodiment is a 3^{rd} embodiment of the holding structure according to the present invention, that preferably depends on the 1^{st} or the 2^{nd} embodiment.

|4a| According to a further preferred embodiment of the holding structure according to the present invention, the holding structure is formed by injection molding from a plastic material, wherein the side walls of the receptacles are inclined at a draft angle γ in relation to longitudinal axis L_{receptable} so that the inner diameter d of the receptacle constantly increases from the bottom end to the upper end. In this context the draft angle γ is preferably in the range from 0.01 to 1 degree, more preferably in the range from 0.025 to 0.75 degrees, even more preferably in the range from 0.05 to 0.5 degrees and most preferably in the range 0.075 to 0.3 degrees. Generally, the draft angle γ is of the order of one degree or less to facilitate the demolding of the holding structure from an injection mold during manufacture by injection molding. According to a particular embodiment of the the holding structure according to the present invention, the side walls of the receptacles are not inclined at all (draft angle γ=0 degree) in at the bottom end of the receptacles, preferably up to a height hₓ in the range from 1 to 5 mm, preferably 2 to 4 mm measured from the bottom end of the receptacles and wherein beginning at the height hₓ the side walls of the receptacles are inclined by the above-mentioned draft angles up to the upper and of the receptacles. Thus, in such an embodiment the inner diameter d of the receptacle is constant in the area from the bottom of the receptacle up to height hₓ and constantly increases from the height hₓ to the upper end. This embodiment is a 4^{th} embodiment of the holding structure according to the present invention, that preferably depends on any of the 1^{st} to the 3^{rd} embodiments.

|5a| According to a further preferred embodiment of the holding structure according to the present invention, the holding structure is formed by injection molding from a plastic material, wherein the holding structure is designed in such a manner that a two-part original mold with a lower mold and an upper mold is used for production by injection molding from the plastic material such that the side walls of the receptacles are not inclined at a draft angle γ in relation to longitudinal axis L_{receptable} such that the inner diameter d of the receptacle constantly increases from the bottom end to the upper end. In this embodiment the inner diameter d_{bottom} of the receptacle at the bottom end either corresponds to the inner diameter dₜₒₚ at the upper end or is even larger than the inner diameter dₜₒₚ at the upper end. This embodiment is a 5^{th} embodiment of the holding structure according to the present invention, that preferably depends on any of the 1^{st} to the 3^{rd} embodiments.

|6a| According to a further preferred embodiment of the holding structure according to the present invention,
the receptacles are arranged in a regular arrangement,
an upper side of the holding structure is formed as a plate-shaped support, and
the receptacles project vertically from the plate-shaped support;
   wherein the side wall is formed as a common partition between each two directly adjacent receptacles of the plurality of receptacles. This embodiment is a 6^{th} embodiment of the holding structure according to the present invention, that preferably depends on any of the 1^{st} to the 5^{th} embodiments.

|7a| According to a further preferred embodiment of the holding structure according to the present invention the partition walls, viewed in a cross-section, are each formed in one piece and without openings. This embodiment is a 7^{th} embodiment of the holding structure according to the present invention, that preferably depends on the 6^{th} embodiment.

|8a| According to a further preferred embodiment of the holding structure according to the present invention, the partition walls, viewed in a cross-section, are formed in two or more separate pieces, wherein the partition wall comprises openings between two neighboured pieces of the partition wall. This embodiment is an 8^{th} embodiment of the holding structure according to the present invention, that preferably depends on the 6^{th} embodiment.

|9a| According to a further preferred embodiment of the holding structure according to the present invention, the receptacles are polygonal or circular in shape when viewed from above. The receptacles may, for example, be hexagonal in shape when viewed in a plan view and arranged directly adjacent to each other in a regular arrangement with hexagonal symmetry. The receptacles may also be octagonal in shape when viewed in a plan view, with four adjacent receptacles arranged in a rhombic arrangement. A particular preferred shape of the receptacles is a rhombic shape. This embodiment is a 9^{th} embodiment of the holding structure according to the present invention, that preferably depends on any of the 1^{st} to the 8^{th} embodiments.

|10a| According to a further preferred embodiment of the holding structure according to the present invention,
A is the surface of the section of the elongated cylindrical body region of the primary packaging container that is accommodated in the receptacle of the holding structure;
B is the part of surface A that can come into contact with a surface of the alignment elements in a given receptacle if the primary packaging container is accommodated in the receptacle in the resting position;
   wherein B/A is less than 0.1, preferably less than 0.05, more preferably less than 0.03, even more preferably less than 0.02 and most preferably less than 0.01. This embodiment is a 10^{th} embodiment of the holding structure according to the present invention, that preferably depends on any of the 1^{st} to the 9^{th} embodiments.

|11a| According to a further preferred embodiment of the holding structure according to the present invention, in each receptacle the alignment elements comprise a plurality of longitudinal alignment elements running in a direction from the upper end to the bottom end of the receptacle and extending over at least 50%, preferably at least 75% and more preferably at least 90% of the entire depth h of the receptable, wherein the alignment elements are adapted and arranged to contact a part of the outer surface of the primary packaging container over at least a certain part of the length of the elongated cylindrical body region that is accommodated in the receptacle. The longitudinal alignment elements running in a direction from the upper end to the bottom end of the receptacle can be designed as continuous alignment elements which are not interrupted on their way from the upper to the lower end. However, they can also consist of several longitudinal elements arranged one behind the other and separated from each other by interruptions Also, in each receptacle there are preferably two or more of these longitudinal alignment elements, more preferably 4 to 6 of these longitudinal alignment elements, that are equally distributed at angular spacings from one another. This embodiment is an 11^{th} embodiment of the holding structure according to the present invention, that preferably depends on any of the 1^{st} to the 10^{th} embodiments.

|12a| According to a further preferred embodiment of the holding structure according to the present invention, in each receptacle there are one more positions Pₓ at which at least two alignment elements are provided with a concave recess the curvature of which corresponds to the outer diameter of the primary packaging container to be accommodated in the receptacle, wherein the alignment elements are adapted and arranged to contact the outer surface of the primary packaging container over at least a part of the circumference of the elongated cylindrical body region that is accommodated in the receptacle. In case of two or more positions Pₓ at which these alignment elements are provided, these positions differ in the height at which they are arranged along longitudinal axis L_{receptable} of the receptacle. Preferably, at least one of these positions Pₓ is located in the upper third, even more preferably in the upper quarter of the receptacle. This embodiment is a 12^{th} embodiment of the holding structure according to the present invention, that preferably depends on any of the 1^{st} to the 10^{th} embodiments.

|13a| According to a further preferred embodiment of the holding structure according to the present invention, in each receptacle there are one more positions Pₓ at which at least two alignment elements are provided, wherein the alignment elements are adapted and arranged to punctually contact the outer surface of the primary packaging container on at least a part of the elongated cylindrical body region that is accommodated in the receptacle. In case of two or more positions Pₓ at which these alignment elements are provided, these positions again differ in the height at which they are arranged along longitudinal axis L_{receptable} of the receptacle. In this context it is also preferred that at a given position Pₓ there is a plurality of punctual alignment elements that are equally distributed at angular spacings from one another. More preferably, the punctual alignment elements are located at least at a first position P₁ in the area of the upper end of the receptacle, preferably in the upper third, more preferably in the upper quarter of the receptacle, and at a second position P₂ in the area of the bottom end of the receptacle, preferably in the lower third, more preferably in the lower quarter of the receptacle. In case of punctual alignment elements that are located at two positions P₁ (in the area of the upper end) and P₂ (in the area of the bottom end) it is also preferred that the alignment elements at position P₁ are located along a first circle located in a first plane and the alignment elements at position P₂ are located along a second circle located in a second plane, the first and the second plane being parallel to each other and being perpendicular to longitudinal axis L_{receptable}, wherein the diameter Ø₁ of the first circle at position P₁ is larger than the diameter Ø₂ of the second circle at position P₂. In this context it is preferred that the term

$$Δ h / \vert {⌀}_{2} - {⌀}_{1} \vert$$

is in the range from 0 to 0.01, more preferably in the range from 0 to 0.007 and most preferably in the range from 0 to 0.0035, Δh being the distance between position P₁ and position P₂ in a direction parallel to longitudinal axis L_{receptable}. This embodiment is a 13^{th} embodiment of the holding structure according to the present invention, that preferably depends on any of the 1^{st} to the 10^{th} embodiments.

|14a| According to a further preferred embodiment of the holding structure according to the present invention, the alignment elements are located on the top of the side walls. This embodiment is a 14^{th} embodiment of the holding structure according to the present invention, that preferably depends on any of the 1^{st} to the 13^{th} embodiments.

|15a| According to a further preferred embodiment of the holding structure according to the present invention, the holding structure further comprises
guide portions adapted and arranged to guide the primary packaging containers as they are inserted into the receptacles, the guide portions being formed as guiding ribs extending in a longitudinal direction of the receptacles,
   wherein the alignment elements are located on the top of the guide portions. In this context it may also be advantageous if lead-in bevels are formed at upper ends of the guide portions which are inclined relative to the guide portions as disclosed in U.S. Pat. No. 11,464,702 B2, wherein the alignment elements may also be located on the top of these lead-in bevels. This embodiment is a 15^{th} embodiment of the holding structure according to the present invention, that preferably depends on any of the 1^{st} to the 13^{th} embodiments.

|1b| A contribution to solving at least one of the objects according to the invention is made by a 1^{st} embodiment of an arrangement comprising:
the holding structure according to the present invention, preferably the holding structure according to any of the 1^{st} to the 15^{th} embodiments;
a plurality of primary packaging containers, each primary packaging container being accommodated in one of the receptacles of the holding structure.
   Preferably, each of the primary packaging containers is, preferably non-destructively, detachably accommodated in one of the receptacles.

|2b| According to a preferred embodiment of the arrangement according to the present invention, the plurality of primary packaging containers comprises a number of primary packaging containers which is in the range from 4 to 500, preferably from 9 to 400, more preferably from 12 to 300, more preferably from 16 to 200, more preferably from 16 to 160, more preferably from 16 to 100, more preferably from 16 to 90, more preferably from 16 to 80, more preferably from 16 to 70, even more preferably from 16 to 60, most preferably from 20 to 160. This embodiment is a 2^{nd} embodiment of the arrangement according to the present invention, that preferably depends on the 1^{st} embodiment.

|3b| According to a further preferred embodiment of the arrangement according to the present invention, each of the primary packaging containers contains a pharmaceutical, medical or cosmetic composition. This embodiment is a 3^{rd} embodiment of the arrangement according to the present invention, that preferably depends on the 1^{st} or the 2^{nd} embodiment.

|4b| According to a further preferred embodiment of the arrangement according to the present invention, each of the primary packaging containers is closed. This embodiment is a 4^{th} embodiment of the arrangement according to the present invention, that preferably depends on any of the 1^{st} to the 3^{rd} embodiments.

|5b| According to a further preferred embodiment of the arrangement according to the present invention, each of the primary packaging containers comprises, in the following sequence along its length,
a) a first end part, comprising a discharge opening,
b) an elongated cylindrical body region, and
c) a further end part.

This embodiment is a 5^{th} embodiment of the arrangement according to the present invention, that preferably depends on any of the 1^{st} to the 4^{th} embodiments.

|6b| According to a further preferred embodiment of the arrangement according to the present invention, the further end part is a standing base, or comprises a further opening, or both. A preferred further orifice is designed to accommodate a plunger. This embodiment is a 6^{th} embodiment of the arrangement according to the present invention, that preferably depends on the 5^{th} embodiment.

|7b| According to a further preferred embodiment of the arrangement according to the present invention, for each of the primary packaging containers, an area of the further opening is more than an area of the discharge opening. This embodiment is an 7^{th} embodiment of the arrangement according to the present invention, that preferably depends on the 6^{th} embodiment.

|8b| According to a further preferred embodiment of the arrangement according to the present invention, the further end part further comprises a rim which projects laterally from the elongated cylindrical body region and at least partially, preferably fully, hems the further opening. This embodiment is a 8^{th} embodiment of the arrangement according to the present invention, that preferably depends on the 6^{th} or the 7^{th} embodiment.

|9b| According to a further preferred embodiment of the arrangement according to the present invention, the first end part of each of the primary packaging containers comprises a connecting element, wherein the connecting element comprises a thread for connecting an auxiliary part to the primary packaging container. A preferred auxiliary part is one selected from the group consisting of a needle, a nozzle, and a tubing, or a combination of at least two thereof. A preferred needle is a hypodermic needle. This embodiment is a 9^{th} embodiment of the arrangement according to the present invention, that preferably depends on any of the 5^{th} to the 8^{th} embodiments.

|10b| According to a further preferred embodiment of the arrangement according to the present invention, the connecting element comprises a thread for connecting an auxiliary part to the respective primary packaging container. This preferred embodiment is an 10^{th} embodiment of the invention, that preferably depends on the 9^{th} embodiment of the invention.

|11b| According to a further preferred embodiment of the arrangement according to the present invention, the first end part of each of the primary packaging containers comprises a male part of a taper fitting. A preferred taper fitting is a Luer taper. Generally, the Luer taper may comprise a thread or not. This embodiment is an 11^{th} embodiment of the arrangement according to the present invention, that preferably depends on any of the 5^{th} to the 10^{th} embodiments.

|12b| According to a further preferred embodiment of the arrangement according to the present invention, the male part of the taper fitting comprises a thread. Preferably, the thread is arranged in a sleeve. This preferred embodiment is a 12^{th} embodiment of the invention, that preferably depends on the 11^{th} embodiment of the invention.

|13b| According to a further preferred embodiment of the arrangement according to the present invention, for each of the primary packaging containers, throughout the elongated cylindrical body region a thickness of a container wall is in a range from +0.3 mm, preferably +0.2 mm, more preferably +0.15 mm, more preferably +0.1 mm, most preferably +0.08 mm, in each case based on a mean value of the thickness of the container wall in the body part of the respective primary packaging container. This embodiment is a 13^{th} embodiment of the arrangement according to the present invention, that preferably depends on any of the 5^{th} to the 12^{th} embodiments.

|14b| According to a further preferred embodiment of the arrangement according to the present invention, for each of the primary packaging containers, throughout the elongated cylindrical body region a thickness of a container wall is in a range from 0.2 to 3 mm, preferably from 0.3 to 2.5 mm, more preferably from 0.4 to 2.2 mm. This embodiment is a 14^{th} embodiment of the arrangement according to the present invention, that preferably depends on any of the 5^{th} to the 13^{th} embodiments.

|15b| According to a further preferred embodiment of the arrangement according to the present invention, throughout the elongated cylindrical body region a thickness of a container wall is in a range from 1.0 to 1.1 mm. In a further preferred embodiment, for each of the primary packaging containers, throughout the elongated cylindrical body region a thickness of a container wall is in a range from 1.4 to 1.8 mm. In yet a further preferred embodiment, for each of the primary packaging containers, throughout the elongated cylindrical body region a thickness of a container wall is in a range from 0.6 to 2.0 mm. This embodiment is a 15^{th} embodiment of the arrangement according to the present invention, that preferably depends on any of the 5^{th} to the 14^{th} embodiments.

|16b| According to a further preferred embodiment of the arrangement according to the present invention, each of the primary packaging containers has a container interior with a volume in a range from 0.5 to 100 ml, preferably from 0.8 to 100 ml, more preferably from 1 to 50 ml, even more preferably from 1 to 10 ml. This embodiment is a 16^{th} embodiment of the arrangement according to the present invention, that preferably depends on any of the 1^{st} to the 15^{th} embodiments.

|17b| According to a further preferred embodiment of the arrangement according to the present invention, the primary packaging containers are selected from the group consisting of vials, syringes, cartridges, and ampoules, or a combination of at least two thereof. A preferred cartridge is designed for being used as a reservoir in a, preferably portable, medical device. A preferred portable medical device is an insulin pump. This embodiment is an 17^{th} embodiment of the arrangement according to the present invention, that preferably depends on any of the 1^{st} to the 16^{th} embodiments.

|18b| According to a further preferred embodiment of the arrangement according to the present invention, each of the primary packaging containers comprises a container wall which at least partially surrounds a container interior, wherein the container wall comprises, preferably consists of, a glass, or a polymer, or both. This embodiment is an 18^{th} embodiment of the arrangement according to the present invention, that preferably depends on any of the 1^{st} to the 17^{th} embodiments.

|19b| According to a further preferred embodiment of the arrangement according to the present invention, the polymer is a cyclic olefin copolymer, or a cycloolefin polymer, or a mixture thereof. This embodiment is a 19^{th} embodiment of the arrangement according to the present invention, that preferably depends on the 18^{th} embodiment.

|20b| According to a further preferred embodiment of the arrangement according to the present invention, the glass is of a type selected from the group consisting of a borosilicate glass, preferably a type I glass; an aluminosilicate glass; and fused silica; or of a combination of at least two thereof. This embodiment is a 20^{th} embodiment of the arrangement according to the present invention, that preferably depends on the 18^{th} embodiment.

|21b| According to a further preferred embodiment of the arrangement according to the present invention, the primary packaging containers have been decontaminated, preferably sterilized. Preferably, the arrangement has been decontaminated, preferably sterilized. In the context of the present application, decontamination is defined as an umbrella term for reducing the amount of microbes and biological agents, such as fungi, bacteria, viruses, spore forms, prions, unicellular eukaryotic organisms, etc. The special terms disinfection and sterilization differ in the amount of reduction of these. While disinfection only reduces the amount of said contaminants, sterilization effectively kills, deactivates, or eliminates all forms of life and other biological agents which are present, i.e., a reduction of 100%. Hence, disinfection is less effective than sterilization. This embodiment is a 21^{st} embodiment of the arrangement according to the present invention, that preferably depends on any of the 1^{st} to the 20^{th} embodiments.

|1c| A contribution to solving at least one of the objects according to the invention is made by a 1^{st} embodiment of a transport unit comprising:
the arrangement according to the present invention, preferably the arrangement according to any of its 1^{st} to the 21^{st} embodiments, and
a secondary packaging container,
   wherein the holding structure and the plurality of primary packaging containers are arranged completely in the secondary packaging container.

|2c| According to a preferred embodiment of the transport unit according to the present invention, a container body of the secondary packaging container comprises
a container opening, and
a container base which is opposite to the container opening in the longitudinal direction.

This embodiment is a 2^{nd} embodiment of the transport unit according to the present invention, that preferably depends on the 1^{st} embodiment.

|3c| According to a further preferred embodiment of the transport unit according to the present invention, the first end parts, or the further end parts of the primary packaging containers face the container opening. This embodiment is a 3^{rd} embodiment of the transport unit according to the present invention, that preferably depends on the 2^{nd} embodiment.

|4c| According to a further preferred embodiment of the transport unit according to the present invention, the secondary packaging container is a tub or tub-shaped. This embodiment is a 4^{th} embodiment of the transport unit according to the present invention, that preferably depends on any of the 1^{st} to the 3^{rd} embodiments.

|5c| According to a further preferred embodiment of the transport unit according to the present invention, the secondary packaging container is closed by a lid. Preferably, the lid is joined to the secondary packaging container. A preferred lid is a multi-layer sheet. Additionally, or alternatively preferred, the lid is gas-permeable. This embodiment is a 5^{th} embodiment of the transport unit according to the present invention, that preferably depends on any of the 1^{st} to the 4^{th} embodiments.

|6c| According to a further preferred embodiment of the transport unit according to the present invention, the transport unit further comprises a, preferably closed, outer packaging, wherein the secondary packaging container is arranged in the outer packaging. A preferred outer packaging is a pouch, preferably made from a plastic film. Additionally, or alternatively preferred, the outer packaging provides a barrier against a permeation of an inert gas. Additionally, or alternatively preferred, the outer packaging is hermetically sealed. Additionally, or alternatively preferred, the outer packaging is less permeable for the inert gas than the lid. Particularly preferred the outer packing provides a barrier action against a permeation of the inert gas, whereas the lid is permeable for the inert gas. This a 6^{th} embodiment of the transport unit according to the present invention, that preferably depends on any of the 1^{st} to the 5^{th} embodiments.

|7c| According to a further preferred embodiment of the transport unit according to the present invention, the outer packaging comprises an atmosphere which comprises an inert gas at a proportion of at least 50 vol.-%, preferably at least 60 vol.-%, preferably at least 70 vol.-%, more preferably at least 80 vol.-%, even more preferably at least 90 vol.-%, most preferably at least 95 vol.-%, in each case based on a volume of the atmosphere. This preferred embodiment is a 7^{th} embodiment of the transport unit according to the present invention, that preferably depends on the 6^{th} embodiment.

|1d| A contribution to solving at least one of the objects according to the invention is made by a use of the holding structure of the invention, preferably of a holding structure according to any of the 1^{st} to 15^{th} embodiments, or of the arrangement of the invention, preferably of the arrangement according to any of the 1^{st} to the 21^{st} embodiments, or of the transport unit of the invention, preferably of the transport unit according to any of the 1^{st} to the 7^{th} embodiments, in each case for storing or transporting the plurality of primary packaging containers. In a preferred embodiment of the use, each of the primary packaging containers contains a pharmaceutical, medical or cosmetic composition.

|1e| A contribution to solving at least one of the objects according to the invention is made by a process for filling primary packaging containers with a pharmaceutical, medical, or cosmetic composition, the process comprising the steps:
A) providing an arrangement according to the present invention, preferably an arrangement according to any of the 1^{st} to the 21^{st} embodiments,
B) filling each primary packaging container accommodated in the receptacles of the holding structure with the liquid pharmaceutical composition in an automated filling machine.

|2e| According to a preferred embodiment of the process for filling primary packaging containers, in the automated filling machine a needle is positioned above the opening of the primary packaging container, followed by an injection of the pharmaceutical, medical, or cosmetic composition through the needle into the container, wherein the primary packaging container is filled with the pharmaceutical, medical, or cosmetic composition following the positioning of the needle above the corresponding primary packaging container. This a 2^{nd} embodiment of the process for filling primary packaging containers according to the present invention, that preferably depends on the 1^{st} embodiment.

|1f| A contribution to solving at least one of the objects according to the invention is made by a use of the holding structure of the invention, preferably of a holding structure according to any of the 1^{st} to 15^{th} embodiments, for holding the plurality of primary packaging containers in a step of filling the primary packaging containers with a pharmaceutical, medical, or cosmetic composition.

### Holding Structure

The holding structure of the invention may, generally, be any device which, for the skilled person, comes into consideration for holding the plurality of primary packaging containers. A preferred holding structure is a carrier structure of a so-called nested solution as they are generally known in the technical field of transport packaging for medical, pharmaceutical and cosmetical primary packaging containers. An example of a known nested solution is commercially available from Schott Pharma AG under the tradename SCHOTT iQ® platform. A preferred holding structure has been prepared by deep drawing or injection molding, where injection molding is particularly preferred. Additionally, or alternatively preferred, the holding structure is made from one or more plastics. Preferably, the plate-shaped carrier element is in one piece with the plurality of receptacles. Further preferably, the holding device is of a one-piece design. Preferably, the receptacles form a regular pattern in a top view on the holding device.

### Alignment Element

The alignment elements serve to prevent, or at least minimise, tilting of the primary packaging containers held in the receptacles of the holding structure. Tilting of the primary packaging containers occurs when an angle other than zero is included between the longitudinal axis L_{container} of the primary packaging container and the longitudinal axis L_{receptacle} of the receptacle that accommodates the primary packaging container (=tilting angle α). The longitudinal axis L_{container} of the primary packaging container is the axis that runs through the center of the elongated cylindrical body region of the primary packaging container, which is preferably rotationally symmetrical about this axis, while the longitudinal axis L_{receptacle} of the receptacle is the axis that runs centrally and perpendicularly through the receptacle in the elongated direction along the depth h of the receptacle.

The alignment elements can have any shape that appears suitable to the person skilled in the art in order to prevent the containers from tilting in the receptacle. For example, they may extend in an elongate direction from the upper to the lower end of the receptacle, with two or more such elongate alignment elements being evenly spaced from one another around the circumference of the receptacle (as shown in FIG. 6). In such an embodiment it is particularly advantageous to apply the alignment elements to the guide elements arranged in a similar manner. Preferably, the internal diameter of the receptacles, which is ultimately defined by the alignment elements, is smaller in the upper area of the receptacles than in the lower area.

According to a further embodiment, the alignment elements can also have concave recesses whose curvature is adapted to the outer radius of the primary packaging containers, so that these alignment elements contact the primary packaging containers at certain positions along at least a part of their circumference (as shown in FIG. 7). Such alignment elements may be arranged locally at one or more positions along the elongate axis of the receptacles, but preferably at least in the upper region of the receptacles, particularly preferably in the upper third, even more preferably in the upper quarter, wherein at a given position Pₓ along the height of the receptacle preferably two such alignment elements arranged opposite one another are provided (as also shown in FIG. 8).

Particularly advantageous, however, are embodiments in which the alignment elements merely enable a point-like contact with the outer surface of the primary packaging containers, such alignment elements also being arranged locally at one or more positions Pₓ along the height of the receptacle, preferably at least in the upper region of the receptacle, particularly preferably in the upper third, even more preferably in the upper quarter, wherein at a given position Pₓ two, three, four, five, six or more such alignment elements arranged in a circular manner and with uniform spacing between each other (as shown in FIG. 3). If the contact is only point-shaped, the friction between the alignment elements and the outer wall of the primary packaging container during insertion and removal of the container can be minimised. In principle, the size of the contact surface between the alignment elements and the primary packaging container, or more precisely between the alignment elements and the elongated cylindrical body region of the primary packaging container, should be as small as possible.

The alignment elements are preferably made of the same material as the holding structure itself. The alignment elements can also already be applied in an injection molding process or in a 3D-printing process used to produce the holding structure themselves, or they can be applied subsequently by 3D-printing, bonding, or welding to corresponding surface areas in the receptacles of the holding structure produced by 3D-printing or injection, molding or deep-drawing.

### Plate-Shaped Carrier Element

The term “plate-shaped” refers to a width and a length of the carrier element each being at least 5 times, preferably at least 10 times, more preferably at least 50 times, most preferably at least 100 times, more than a thickness of the carrier element. Preferably, the thickness of the carrier element is in the range from 0.5 to 5 mm, more preferably from 0.5 to 3 mm, even more preferably from 0.5 to 2 mm, most preferably from 0.8 to 2 mm. Additionally or alternatively preferred, the carrier element has an essentially flat top surface or an essentially flat bottom surface or both. Here, the term “plate-shaped carrier element” refers to an essentially flat element without any optional macroscopic protrusions on the top or bottom surface of the carrier element. Preferably, the top and bottom surfaces of the plate-shaped carrier element are essentially rectangular. Here, “essentially” means that the top or bottom surfaces may have rounded corners and/or recesses at a rim area.

### Side Wall of the Receptacle

The receptacle wall of a receptacle, preferably, consists of all wall elements of the receptacle. Here, the receptacle wall may enclose the receptacle interior only partially in the sense that the wall body has the first and further openings. The wall body is the geometric body which is formed by the receptacle wall.

### Guide Portions

The guiding portions serve to guide the containers into the receptacles during insert. Preferably, these guiding portions are formed as guiding ribs extending in a longitudinal direction of the receptacles, wherein it is preferred that in each receptable two or more of these guiding ribs are distributed at certain angular spacings from one another. Preferably, these guiding portions are not equally distributed with identical angular spacings between each other within a given receptacle.

It may be sufficient if the guiding ribs protrude radially inwards from side walls or side wall portions of the receptacles only to a relatively small extent, for example by a distance that can be of the order of one millimetre or less only. The guiding ribs or at least their front edges may be designed as planar, inclined surfaces.

The guiding ribs can extend continuously over almost the entire depth h of the receptable. Such guiding ribs are preferably inclined at an angle β of inclination relative to longitudinal axis L_{receptacle} of the receptacle, wherein β is preferably in the range from 0.01 to 1 degrees, more preferably in the range from 0.025 to 0.75 degrees, even more preferably in the range from 0.05 to 0.5 degrees and most preferably in the range 0.075 to 0.3 degrees. Generally, the angle of inclination of the guiding ribs relative to longitudinal axis L_{receptacle} may be negligible in principle or it is of the order of one degree or less to facilitate the demolding of the holding structure from an injection mold during manufacture by injection molding.

In the alternative, the guiding ribs may also comprise separate upper portions near the upper ends of the receptacles and lower guiding portions near the lower ends of the receptacles as disclosed, for example, in U.S. Pat. No. 11,286,095 B2.

### Primary Packaging Container

A primary packaging container is the container that most closely protects the product in its distribution channels. The primary packaging container can also be referred to as retail or consumer packaging container. It follows that a test tube is not a primary packaging container.

The primary packaging containers may have any size or shape which the skilled person deems appropriate in the context of the invention. A preferred primary packaging container is a primary packaging container for a medical, pharmaceutical or cosmetical composition. Preferably, the primary packaging container is suitable for packaging parenteralia in accordance with section 3.2.1 of the European Pharmacopoeia, 7^{th} edition from 2011. A particularly preferred primary packaging container is a vial, syringe, cartridge or ampoule.

Preferably, each of the primary packaging containers comprises, in the following sequence along its length a first end part, comprising a discharge orifice; an elongated cylindrical body region; and a further end part. Preferably, a first end part of the primary packaging container comprises a discharge orifice, which allows for discharging a medical, pharmaceutical or cosmetical composition from the primary packaging container interior of the primary packaging container. In that case, the container wall of the primary packaging container encloses the container interior only partially. A preferred primary packaging container, the first end part of which comprises a discharge orifice is a vial, a syringe or a cartridge. A preferred primary packaging container, the first end part of which does not comprise a discharge orifice is an ampoule. In that case, the container wall of the primary packaging container entirely encloses the container interior. Additionally, or alternatively preferred, the further end part is a standing base, or comprises a further orifice, or both. In the case of a further orifice, the primary packaging container, preferably, is a syringe. In the case of a standing base, the primary packaging container, preferably, is a vial, cartridge, or ampoule. For a preferred primary packaging container, the elongated cylindrical body region follows the first end part a shoulder. This preferred primary packaging container may be a vial, syringe, cartridge, or ampoule, wherein a vial, cartridge or ampoule is preferred. Additionally, or alternatively preferred, the further end part follows the elongated cylindrical body region via a heel. In that case, the primary packaging container, preferably, is a vial, cartridge or ampoule. Preferably, the elongated cylindrical body region is a lateral region of the primary packaging container. Particularly preferable, the elongated cylindrical body region of the container wall forms a hollow cylinder. In case of a syringe, the elongated cylindrical body region is often referred to as barrel. Additionally, or alternatively preferred, the first end part comprises, more preferably consists of, from top to bottom of the primary packaging container a flange and a neck. In this case, the primary packaging container, preferably, is a vial, cartridge or ampoule.

The primary packaging container is preferably a glass container, a wall of glass (container wall) of which at least partially encloses a container interior of the primary packaging container. Preferably, the wall of glass is of a one-piece design. The wall of glass may preferably be made by blow molding a glass melt; or by preparing a tube of a glass, preferably in form of a hollow cylinder, optionally forming the bottom of the primary packaging container from one end of the tube, thereby closing the tube at this end, and forming the top region of the primary packaging container from the opposite end of the tube. Preferably, the wall of glass is transparent. Alternatively, the container wall is preferably made from a polymer. In that case, the container wall is also preferred to be transparent.

For the use in this document, the interior volume of the primary packaging container interior represents the full volume of the interior of the container. This volume may be determined by filling the interior of the container with water up to the brim and measuring the volume of the amount of water which the interior can take up to the brim. Hence, the interior volume of the container interior as used herein is not a nominal volume as it is often referred to in the technical field of pharmacy. This nominal volume may for example be less than the interior volume by a factor of about 0.5.

### Glass

The container wall of each of the primary packaging containers, preferably, comprises a glass, more preferably essentially consists of the glass. This glass may be any type of glass and may have any composition which the skilled person deems suitable in the context of the invention. Preferably, the glass is suitable for pharmaceutical packaging. Particularly preferable, the glass is of type I in accordance with the definitions of glass types in section 3.2.1 of the European Pharmacopoeia, 7^{th} edition from 2011. Additionally, or alternatively preferable to the preceding, the glass is selected from the group consisting of a borosilicate glass, an aluminosilicate glass, and fused silica; or a combination of at least two thereof, wherein a borosilicate glass is particularly preferred. For the use in this document, a borosilicate glass is a glass which has a content of B₂O₃ of at least 1 wt.-%, preferably at least 2 wt.-%, more preferably at least 3 wt.-%, more preferably at least 4 wt.-%, even more preferably at least 5 wt.-%, particularly preferable in a range from 5 to 15 wt.-%, in each case based on the total weight of the glass. A preferred borosilicate glass has a content of Al₂O₃ of less than 7.5 wt.-%, preferably less than 6.5 wt.-%, particularly preferably in a range from 0 to 5.5 wt.-%, in each case based on the total weight of the glass. In a further aspect, the borosilicate glass has a content of Al₂O₃ in a range from 3 to 7.5 wt.-%, preferably in a range from 4 to 6 wt.-%, in each case based on the total weight of the glass.

### Medical, Pharmaceutical and Cosmetical Compositions

In the context of the invention, every medical composition, every pharmaceutical composition and every cosmetic composition, which the skilled person deems suitable, comes into consideration. A medical composition is a composition for the use in a medical treatment. A medical composition does not necessarily comprise an active ingredient. A pharmaceutical composition is a composition comprising at least one pharmaceutically active ingredient. A preferred pharmaceutically active ingredient is a vaccine. A cosmetic composition is a composition comprising at least one cosmetically active ingredient. A preferred cosmetically active ingredient is hyaluronic acid or botulinum toxin. The medical, pharmaceutical or cosmetical composition may be fluid or solid or both, wherein a fluid composition is particularly preferred herein. A preferred solid composition is granular such as a powder, a multitude of tablets or a multitude of capsules. A further preferred medical, pharmaceutical or cosmetical composition is a parenterialium, i.e. a composition which is intended to be administered via the parenteral route, which may be any route which is not enteral. Parenteral administration can be performed by injection, e.g. using a needle (usually a hypo-dermic needle) and a syringe, or by the insertion of an indwelling catheter.

### Secondary Packaging Container

The secondary packaging container may, generally, be any container which, for the skilled person, comes into consideration for accommodating the holding structure, preferably the arrangement. A preferred secondary packaging container is a tub. Particularly preferable, the secondary packaging container is a tub of a so-called nested solution as they are generally known in the technical field of transport packaging for medical, pharmaceutical and cosmetical containers. An example of a known nested solution is commercially available from Schott AG under the tradename SCHOTT iQ® platform. A preferred secondary packaging container has been prepared by deep drawing or injection molding, where deep drawing is particularly preferred. Additionally, or alternatively preferred, the secondary packaging container is made from one or more plastics. In this context, a preferred plastic is one, selected from the group, consisting of a polycondensation polymer, preferably polyethylene terephthalate; a polyacrylate, preferably polymethylmethacrylate; and a polyolefin, preferably polypropylene or polyethylene; a polystyrene or a combination of at least two thereof.

### Directions

The lateral and longitudinal directions, as referred to herein, are perpendicular to one another. The longitudinal direction, preferably, extends along the lengths of the receptacles. Preferably, any direction, which is perpendicular to the longitudinal direction comes into consideration as lateral direction. Thus, a multitude of lateral directions may derive from a single longitudinal direction. Here, the lateral directions, preferably, form a plane which is perpendicular to the longitudinal direction. Preferably, this plane is a plane of plate-like extension of the plate-shaped carrier element. Any transversal section, herein, is perpendicular to the longitudinal direction.

Unless otherwise specified in the description or the particular figure:

### BRIEF DESCRIPTION OF THE DRAWINGS

FIG. 1A is a cross-sectional view of a primary packaging container 200 accommodated in a receptacle 101 of a holding structure 100 according to the present invention in the resting position;

FIG. 1B shows a primary packaging container 200 that can be accommodated in the receptacles 101 of the holding structure 100 according to the present invention;

FIG. 2 is a cross-sectional view of a primary packaging container 200 accommodated in a receptacle 101 of a holding structure 100 according to the present invention in the lifting position;

FIG. 3 is a top view of a holding structure 100 according to the present invention in which the alignment elements 106 are attached to the side walls 104 of the receptacles 101;

FIG. 4 is a further top view of a holding structure 100 according to the present invention in which the alignment elements 106 are located on the top of the guide portions 109;

FIG. 5 is a cross-sectional view of a receptacle 101 in which the side walls 104 of the receptacles 101 are inclined at a draft angle γ;

FIG. 6 is a cross-sectional view of a receptacle 101 in which the alignment elements 106 are adapted and arranged to contact a part of the outer surface of the primary packaging containers 200 over at least a certain part of the length of the section 201′ of the primary packaging container 200 that is accommodated in the receptacle 101;

FIG. 7 is the top view of the receptacle 101 shown in FIG. 6;

FIG. 8A is a cross-sectional view of a receptacle 101 in which the alignment elements 106 are provided with a concave recess 108 the curvature of which corresponds to the outer diameter of the primary packaging container 200 to be accommodated in the receptacle 101;

FIG. 8B is a top view of the alignment elements 106 in the receptacle shown in FIG. 8A;

FIG. 9 is a cross-sectional view of a receptacle 101 in which the alignment elements 106 are adapted and arranged to punctually contact the outer surface of the primary packaging container 200 accommodated in the receptacle 101 at position Pₓ;

FIG. 10 is a side view of a receptacle 101 in a holding structure 100 according to the present invention that is designed in such a manner that a two-part original mold with a lower mold 110 and an upper mold 111 is used for production of the holding structure 101;

FIG. 11A light microscope image of a part of the outer surface of the container wall from the Comparative Example;

FIG. 11B light microscope image of a part of the outer surface of the container wall from the Example.

### DETAILED DESCRIPTION

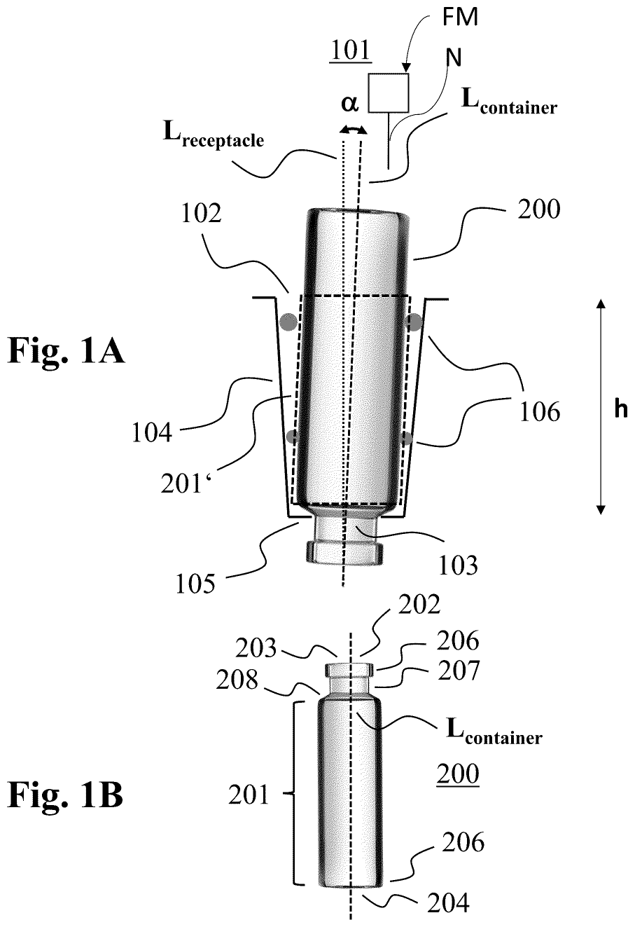

FIG. 1A is a cross-sectional view of a primary packaging container 200 accommodated in a receptacle 101 of a holding structure 100 according to the present invention in the resting position. The receptacle 101 serves to receive the primary packaging containers 200 therein at least in sections 201′. Each receptacle 101 has a depth h, an upper end 102 for inserting the primary packaging container 200 into the receptacle 101, a bottom end 103 having a holding portion 104 configured to limit an axial movement of the primary packaging container 200 in the receptacle 101, a circumferentially formed side wall 105 extending in a longitudinal direction from the upper end 102 to the bottom end 103 and a longitudinal axis L_{receptable} that runs in a longitudinal direction through the center of the receptacle 101. In FIG. 1A the section 201′ of the elongated cylindrical body region 201 of the primary packaging container 200 that in the resting position is accommodated in the receptacle is marked by a dashed parallelogram. As shown in FIG. 1A, the holding structure according to the present invention each receptacle 101 comprises alignment elements 106 (in FIG. 1A shown in the form of grey circles) that project radially into the receptacle 101 and that are adapted and arranged to contact a part of the outer surface of the elongated cylindrical body region 201 of a primary packaging container 200 accommodated in the receptacle 101. The alignment elements 106 in each receptacle 101 are adapted and arranged to contact a part of the outer surface of the elongated cylindrical body region 201 in such a way that a primary packaging container 200 accommodated in the receptacle 101 in the resting position is tilted by an angle α of not more than 2.4 degrees, wherein the tilting angle α is the angle that is formed between longitudinal axis L_{receptacle} and longitudinal axis L_{container} of the primary packaging container 200 accommodated in the receptacle 101. The resting position is the position in which the holding portion 104 limits the axial movement of the primary packaging container 200 accommodated in the in the receptacle 101. The resting position is thus the position in which the primary packaging container 200 is accommodated up to the maximum depth in the receptacle 101. As can be seen, in the receptacle 101 shown in FIG. 1A in the resting position the holding portions come into contact with a shoulder 208 of the primary packaging container 200 (see shoulder 208 in FIG. 1B).

FIG. 1B shows a primary packaging container 200 in the form of a vial that can be accommodated in the receptacles 101 of the holding structure 100 according to the present invention. The primary packaging container 200 may comprise a container wall which partially surrounds a container interior. The container wall forms, in the following sequence from top to bottom, a first end part 202, comprising a discharge orifice 203, an elongated cylindrical body region 201, and a further end part 204. The elongated cylindrical body region 201 is a preferably hollow cylinder. The further end part 204 comprises a standing base 205. Besides the discharge orifice 203, the first end part 202 consists of a flange 206 and a neck 207. The elongated cylindrical body region 201 follows the first end part 202 via the shoulder 208. The further end part 204 follows the elongated cylindrical body region 201 via a heel 209. The container wall is made from type I borosilicate glass. FIG. 1A also solely schematically shows an automated filling machine FM with a needle N for filling the containers 200.

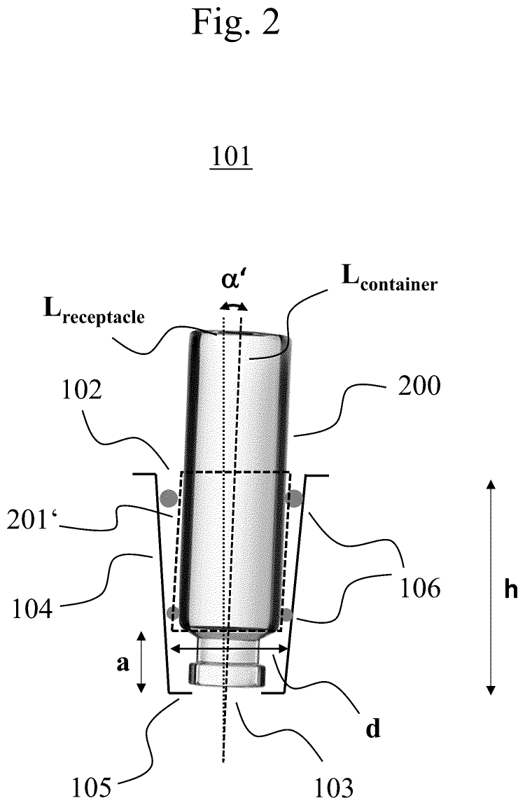

FIG. 2 is a cross-sectional view of a primary packaging container 200 accommodated in a receptacle 101 of a holding structure 100 according to the present invention in the lifting position. The lifting position is the position in which the primary packaging container 200 is lifted out of the resting position in a direction away from the bottom end 103 to a lifting height a, wherein a is 10 to 50%, preferably 15 to 45% and more preferably 20 to 40% of the depth h of the receptacle. Such a lifting of primary packaging containers in a holding structure occurs, for example, if in an automated filling process optical sensors are used to confirm the correctness of the filling height. As can be seen in FIG. 2, the alignment elements 106 preferably also ensure that even in the lifting position the tilting angle α′ does not exceed a certain maximum value.

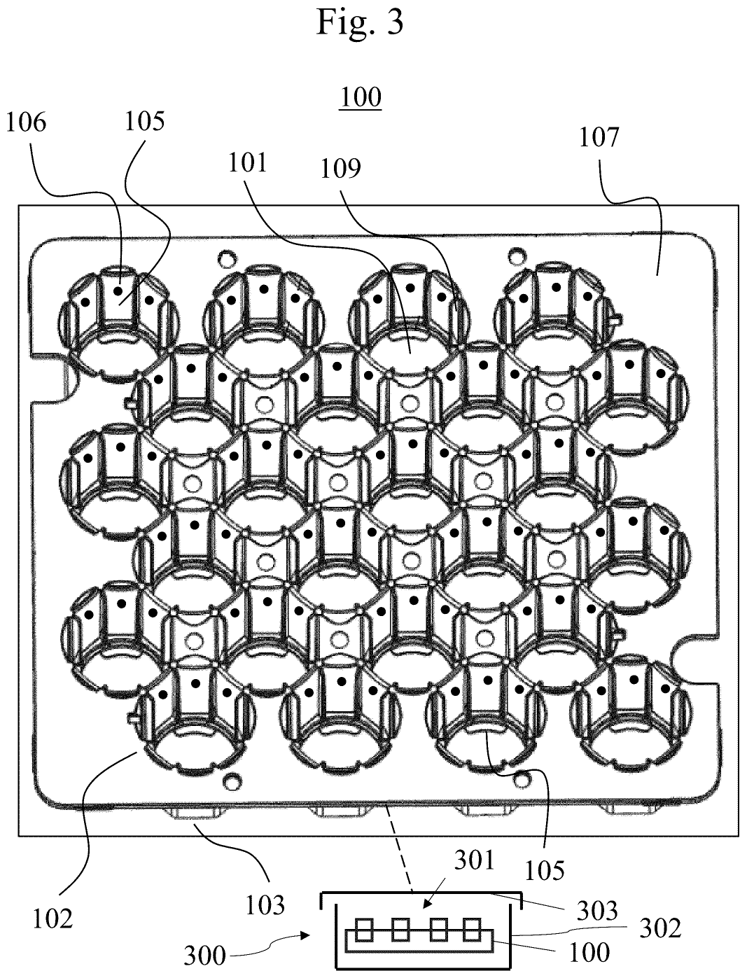

FIG. 3 is a top view of a holding structure 100 according to the present invention in which the alignment elements 106 are attached to the side walls 104 of the receptacles 101. As can be seen, the receptacles 101 are arranged in a regular arrangement. An upper side of the holding structure 100 is formed as a plate-shaped support 107 and the receptacles 101 project vertically from the plate-shaped support 107. The side wall 104 of each receptacle 101 is formed as a common partition between each two directly adjacent receptacles 101 of the plurality of receptacles 101. As can also be seen in FIG. 3, holding protrusions 105 acting as holding portions 105 are provided at the bottom ends 103 of the receptacles 101, which extend radially inwards into the receptacles 101. Each receptacle 101 may, for example, have two holding protrusions 105 which are diametrically opposite to each other. In the holding structure 100 shown in FIG. 3 the alignment elements 106 are provided as point-engaging contact elements 106 in the upper area of the receptacle 101. Also shown in FIG. 3 solely schematically in cross section is a secondary container 302 with a lid 303. Holding structure 100 forms with the primary packaging containers an arrangement 301 which fits in the secondary container 302 to define a transport unit 300.

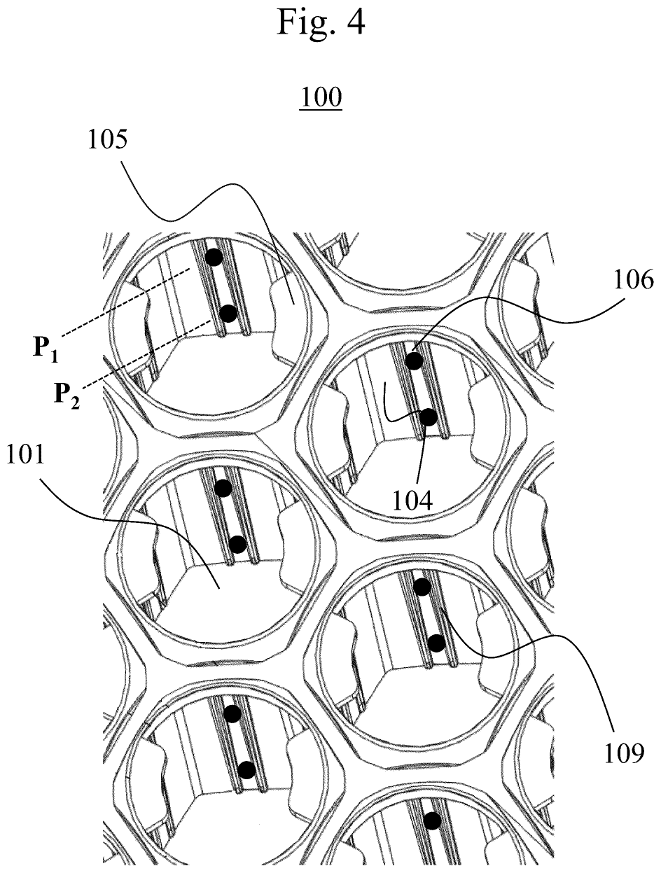

FIG. 4 is a further top view of a holding structure 100 according to the present invention in which the alignment elements 106 are provided as point-engaging contact elements 106 that are located on the top of the guide portions 109 in the receptacles 101. The guide portions 109 are adapted and arranged to guide the primary packaging containers 200 as they are inserted into the receptacles 101. In the holding structure 100 shown in FIG. 4 the guide portions 109 are formed as guiding ribs 109 extending in a longitudinal direction of the receptacles 101.

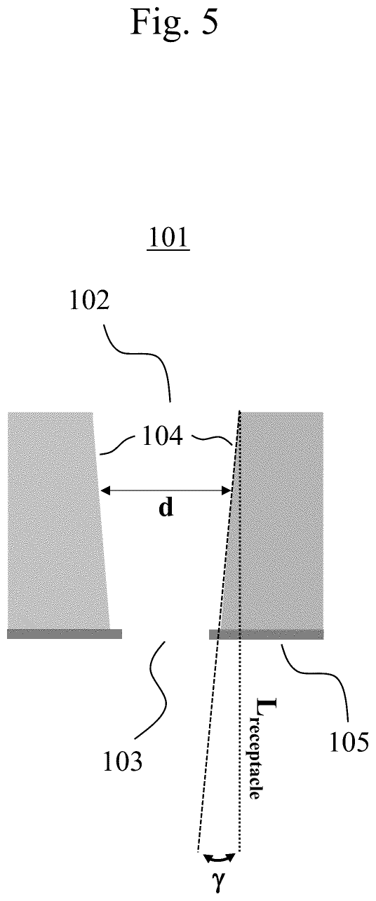

FIG. 5 is a cross-sectional view of a receptacle 101 in which the side walls 104 of the receptacles 101 are inclined at a draft angle γ so that the inner diameter d of the receptacle 101 constantly increases from the bottom end 103 to the upper end 102. In this context the draft angle γ is preferably in the range from 0.01 to 1 degree, more preferably in the range from 0.025 to 0.75 degrees, even more preferably in the range from 0.05 to 0.5 degrees and most preferably in the range 0.075 to 0.3 degrees.

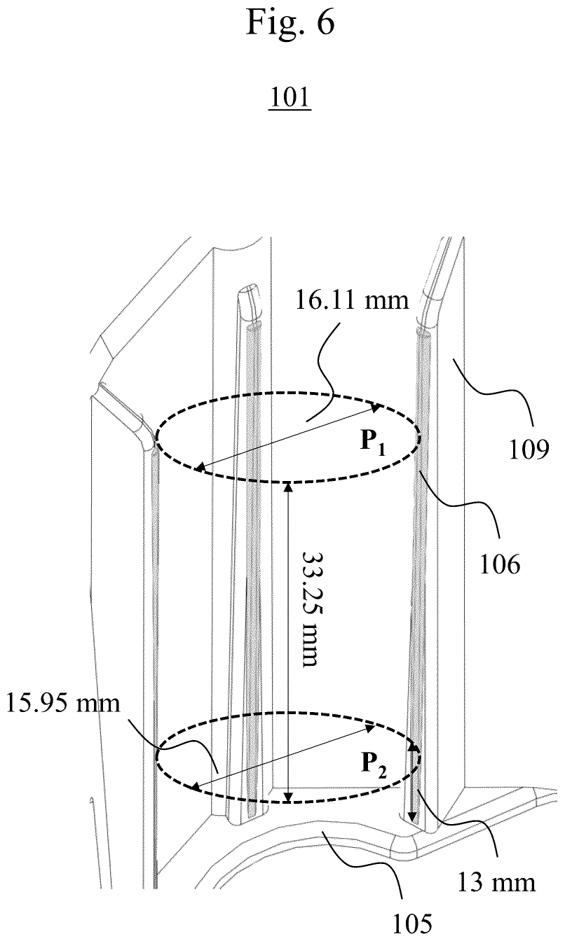

FIG. 6 is a cross-sectional view of a receptacle 101 in which the alignment elements 106 are adapted and arranged to contact a part of the outer surface of the primary packaging containers 200 over at least a certain part of the length of the elongated cylindrical body region 201 that is accommodated in the receptacle 101. In the embodiment shown in FIG. 6, these longitudinal alignment elements 106 are located on the top of the guiding ribs 109 and extend over almost the entire length of these guiding ribs 106.

FIG. 7 is the top view of a part of a holding structure 100 according to the present invention as shown in FIG. 6 in which alignment elements 106 that contact the outer surface of the primary packaging containers 200 accommodated in the receptacle 101 are located on top of the surface of guiding ribs 109 like the arrangement in FIG. 6. In the receptacle 101 shown in FIGS. 6 and 7, the inner diameter of the receptacle 101 as defined by the dimensions of the alignment elements 106 as determined in a position P₂ in a height of 13 mm above the bottom end 103 of the receptacle 101 is 15.95 mm, whereas the inner diameter of the receptacle 101 as defined by the dimensions of the alignment elements 106 determined at a position P₁ in a distance of 33.25 mm from position P₂ is 16.11 mm.

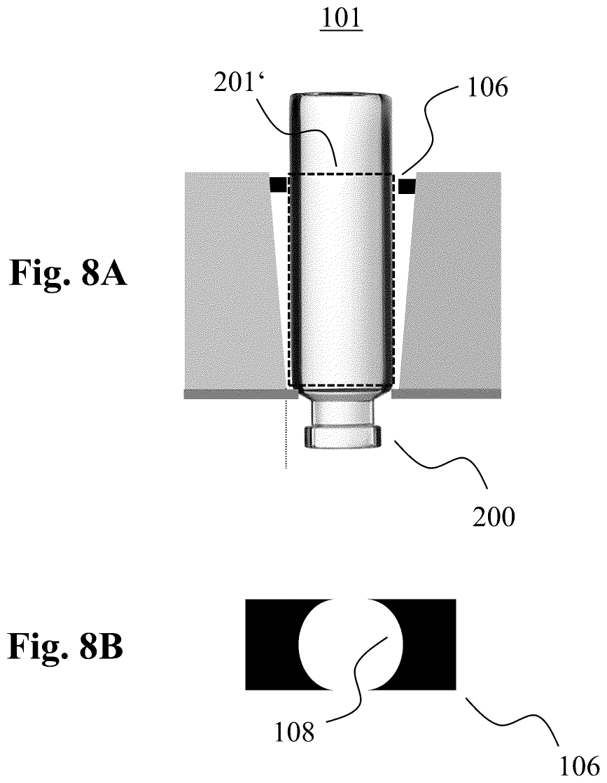

FIG. 8A is a cross-sectional view of a receptacle 101 in which the alignment elements 106 are provided with a concave recess 108 the curvature of which corresponds to the outer diameter of the primary packaging container 200 to be accommodated in the receptacle 101. In the embodiment shown in FIG. 8A, the receptacle 101 comprises such alignment elements only in the upper area close to the upper end 102 of the receptacle, wherein—as shown in FIG. 8 B—two such alignment elements 106 may be arranged opposite each other in such a way that their concave recesses 108 together form a circular recess in which the elongated cylindrical body region 201′ of the primary packaging container 200 is enclosed.

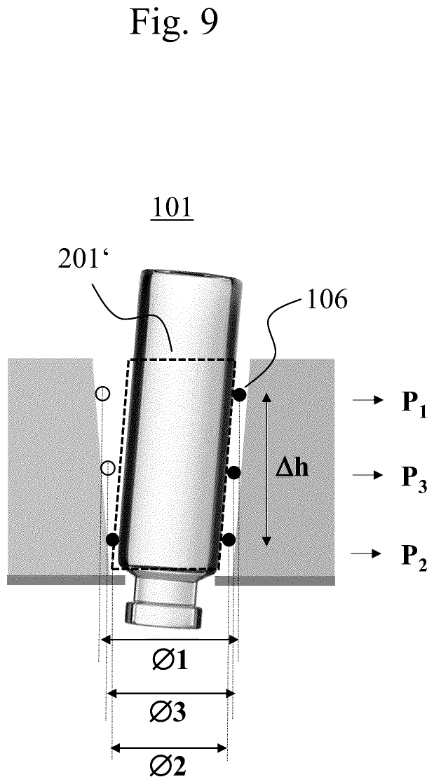

FIG. 9 is a cross-sectional view of a receptacle 101 in which the alignment elements 106 are adapted and arranged to punctually contact the outer surface of the primary packaging container 200 accommodated in the receptacle 101 at position Pₓ. In the embodiment shown in FIG. 8, these point-engaging contact elements 106 may be localized in three different heights within a given receptacle, indicated as positions P₁ near the upper end 102 of the receptacle 101, P₂ near the bottom end 103 of the receptacle 101 and P₃ located between P₁ and P₂. The alignment elements at position P₁ are preferably located along a first circle located in a first plane and the alignment elements at position P₂ are located along a second circle located in a second plane, the first and the second plane being parallel to each other and being perpendicular to longitudinal axis L_{receptable}, wherein the diameter Ø₁ of the first circle at position P₁ is larger than the diameter Ø₂ of the second circle at position P₂. In case of a receptacle 101 in which the alignment elements 106 are located at three different positions P₁, P₂ and P₃ as shown in FIG. 8, the alignment elements 101 at position P₃ are preferably also located along a third circle having a diameter Ø₃, wherein Ø₁>Ø₃>Ø₂.

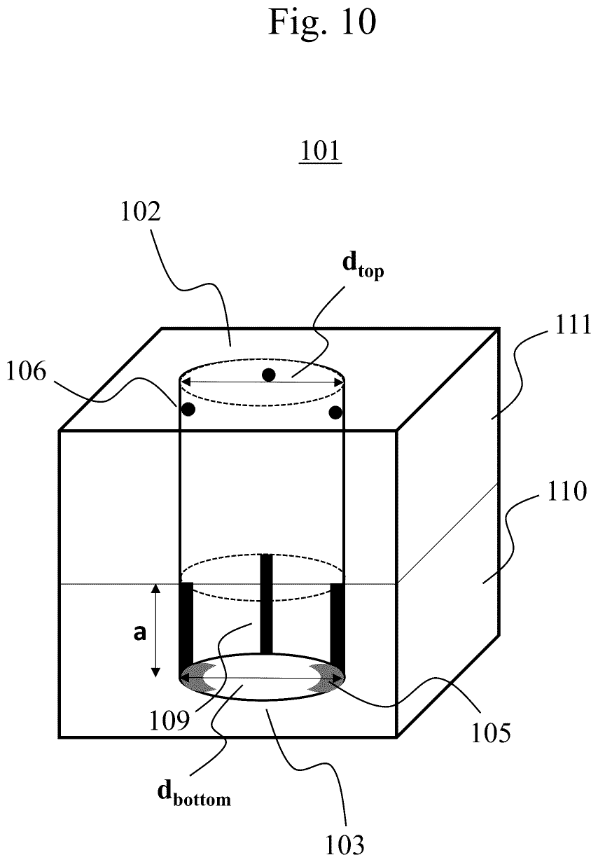

FIG. 10 is a side view of a receptacle 101 in a holding structure 100 according to the present invention that is designed in such a manner that a two-part original mold with a lower mold 110 and an upper mold 111 is used for production of the holding structure 101, wherein the alignment elements 106 are located in part of the receptacle formed by the upper mold 111. Using such a two-component mold allows the provision of receptacles 101 the side walls of which are not inclined at all so that the inner diameter d_{bottom} of the receptacle at the bottom end corresponds to the inner diameter dₜₒₚ at the upper end. Such a receptacle allows the accommodation of primary packaging containers with a particularly low tilting angle α even if the containers are lifted in a lifting height a.

### Test Methods

For the determination of the abrasion of the outer surface of the primary packaging containers in the form of a cartridge inserted into the receptacle of a holding structure the cartridge was first tempered for 24 hours at 250° C. in an oven. After the cartridges had cooled down to around 24° C., they were manually inserted into a nest with receptacles comprising guiding ribs and removed by actively rubbing the cartridges along these guiding ribs. The outer surface of the cartridges was then visually inspected using light microscopy.

### EXAMPLE

In a holding structure as shown in FIG. 6 comprising receptacles with a draft angle γ of 0.2 degrees the guiding ribs were modified by attaching thereto within each receptacle alignment elements such that the inner diameter defined by these alignment elements is 15.95 mm at a position P₂ 13 mm above the bottom end of the receptacle and 16.11 mm at a position P₁. The vertical distance between heights P₁ and P₂ is 33.25 mm and the tilting angle α is 1.16 degrees.

### COMPARATIVE EXAMPLE

The same holding structure was used as in the Example, with the sole difference that the guiding ribs were not modified with any alignment elements.

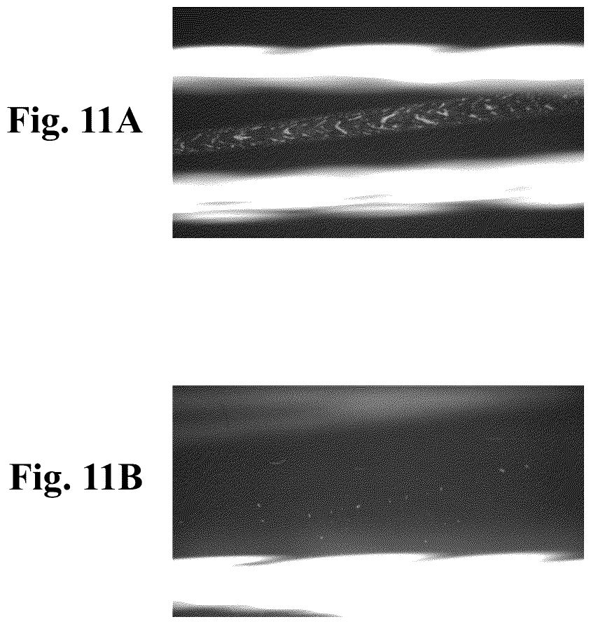

As can be seen in FIGS. 11A and 11B, the alignment elements help to significantly reduce the abrasion of the outer surface of the cartridges when they are moved inside the receptacles.

### LIST OF REFERENCE NUMBERS

100 holding structure
101 receptacle
102 upper end of receptacle 101
103 bottom end of receptacle 101
104 side wall of receptacle 101
105 holding portion of receptacle 101
106 alignment elements
107 plate-shaped carrier element
108 concave recess
109 guide portions
110 lower mold
111 upper mold
200 primary packaging container
201 elongated cylindrical body region of the primary packaging container 200
201′ section of the elongated cylindrical body region 201 of the primary packaging container 200 that is accommodated in the receptacle 101
202 first end part of the primary packaging container 200
203 discharge orifice
204 further end part of the primary packaging container 200
205 standing base
206 flange
207 neck
208 shoulder
300 transport unit according to the invention
301 arrangement according to the invention
302 secondary packaging container
303 lid

What is claimed is:

1. A holding structure for concurrently holding a plurality of primary packaging containers for substances for pharmaceutical, medical or cosmetic applications, each primary packaging container including an elongated cylindrical body region having a container longitudinal axis running in a container longitudinal direction through a center of the elongated cylindrical body region, the holding structure comprising:
a plurality of receptacles for accommodating at least a section of the elongated cylindrical body region of the primary packaging container, each receptacle having a depth, an upper end for inserting the primary packaging container into the receptacle, a bottom end having a holding portion configured to limit an axial movement of the primary packaging container in the receptacle, a circumferentially formed side wall extending in a longitudinal direction from the upper end to the bottom end and a longitudinal axis running in the longitudinal direction through a center of the receptacle;
alignment elements in each receptacle projecting radially into the receptacle and adapted and arranged to contact a part of the outer surface of the elongated cylindrical body region of a primary packaging container accommodated in the receptacle;
wherein the alignment elements in each receptacle are adapted and arranged to contact a part of the outer surface of the elongated cylindrical body region in such a way that a primary packaging container accommodated in the receptacle in a resting position is tilted by an angle of not more than 2.4 degrees, the angle being formed between the longitudinal axis and the container longitudinal axis and the resting position being a position where the holding portion limits the axial movement of the primary packaging container accommodated in the in the receptacle.

2. The holding structure as recited in claim 1 wherein the alignment elements in each receptacle are adapted and arranged to contact a part of the outer surface of the elongated cylindrical body region in such a way that a primary packaging container accommodated in the receptacle in a lifting position is still tilted by a lifted tilting angle of not more than 2.4 degrees, the lifted tilting angle being formed between the longitudinal axis and the container longitudinal axis of the primary packaging container accommodated in the receptacle and the lifting position being where the primary packaging container is lifted out of the resting position in a direction away from the bottom end to a lifting height 10 to 50% of the depth of the receptacle.

3. The holding structure as recited in claim 1 wherein the holding structure is formed by injection molding from a plastic material and wherein the side walls of the receptacles are inclined at a draft angle in relation to the longitudinal axis so that an inner diameter of the receptacle constantly increases from the bottom end to the upper end.

4. The holding structure as recited in claim 1 wherein the holding structure is formed by injection molding from a plastic material, wherein the holding structure is designed in such a manner that a two-part original mold with a lower mold and an upper mold is used for production by injection molding from the plastic material such that the side walls of the receptacles are not inclined at a draft angle in relation to the longitudinal axis so that an inner diameter of the receptacle at the bottom end corresponds to the inner diameter at the upper end.

5. The holding structure as recited in claim 1 wherein
the receptacles are arranged in a regular arrangement,
an upper side of the holding structure is formed as a plate-shaped carrier element, and
the receptacles project vertically from the plate-shaped carrier element;
wherein the side wall is formed as a common partition between each two directly adjacent receptacles of the plurality of receptacles.

6. The holding structure as recited in claim 1 wherein in each receptacle the alignment elements include a plurality of longitudinal alignment elements running in a longitudinal direction from the upper end to the bottom end of the receptacle and extending over at least 50% of an entirety of the depth of the receptable.

7. The holding structure as recited in claim 1 wherein in each receptacle there are one more positions where at least two alignment elements are provided with a concave recess, a curvature of the concave recess corresponding to an outer diameter of the primary packaging container to be accommodated in the receptacle.

8. The holding structure as recited in claim 1 wherein in each receptacle there are one more positions where at least two alignment elements are provided, wherein the alignment elements are adapted and arranged to punctually contact the outer surface of the primary packaging container on at least the part of the elongated cylindrical body region accommodated in the receptacle.

9. The holding structure as recited in claim 1 wherein the alignment elements are located at a top of the side walls.

10. The holding structure as recited in claim 1 further comprising
guide portions adapted and arranged to guide the primary packaging containers as they are inserted into the receptacles, the guide portions being formed as guiding ribs extending in the longitudinal direction;
wherein the alignment elements are located at a top of the guide portions.

11. An arrangement comprising:
the holding structure as recited in claim 1;
a plurality of primary packaging containers, each of the primary packaging containers being accommodated in one of the receptacles of the holding structure.

12. A transport unit comprising:
the arrangement as recited in claim 11; and
a secondary packaging container,
wherein the holding structure and the plurality of primary packaging containers are arranged completely in the secondary packaging container.

13. A process for filling primary packaging containers with a pharmaceutical, medical, or cosmetic composition, the process comprising the steps:
A) providing the arrangement as recited in claim 11;
B) filling each a primary packaging containers accommodated in the receptacles of the holding structure with the composition in liquid form in an automated filling machine.

14. The process as recited in claim 13 wherein in the automated filling machine a needle is positioned above the opening of the primary packaging container, followed by an injection of the pharmaceutical, medical, or cosmetic composition through the needle into the container, wherein the primary packaging container is filled with the pharmaceutical, medical, or cosmetic composition following the positioning of the needle above the corresponding primary packaging container.
